(19) United States
(12) Patent Application Publication
(10) Pub. No.: US 2006/0128718 A1
(43) Pub. Date: Jun. 15, 2006

(54) Tetrahydropyridazine derivatives having a pesticidal effect

(76) Inventors: Rainer Fuchs, Wuppertal (DE); Fritz Maurer, Lauffen (DE); Jorg Konze, Koln (DE)

Correspondence Address: BAYER CROPSCIENCE LP;Patent Department, 100 BAYER ROAD, PITTSBURGH, PA, 15205-9741, US

(21) Appl. No.: 10/526,166

(22) PCT Filed: Aug. 18, 2003
(86) PCT No.: PCT/EP03/09122

§ 371 Date: Dec. 12, 2005

(30) Foreign Application Priority Data
Aug. 28, 2002 (DE) 102 39 480.6

(51) Int. Cl. A61K 31/501; C07D 403/04

(52) U.S. Cl. 514/252.05; 544/238

(57) ABSTRACT

This invention relates to novel tetrahydropyridazine derivatives of the formula (I) 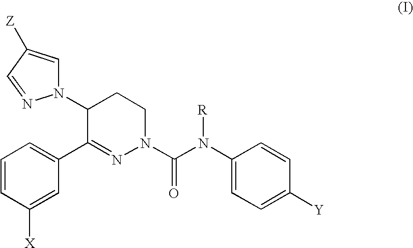 wherein R, X, Y and Z have the meanings outlined in the disclosure, to multiple processes for preparing these materials and to their use to control animal pests.

The present invention relates to novel tetrahydropyridazine derivatives, a process for the preparation of such and their use as pesticides.

It is known that certain tetrahydropyridazine carboxamides exhibit good efficacy against animal pests (cf. e.g. DE-A 43 03 658, WO 91/17983, and DE-A 101 36 066).

However, the spectrum or duration of action of these prior art compounds is not completely satisfactory for all areas of application, especially for certain organisms or at low application concentrations.

Novel tetrahydropyridazine derivatives of the formula (I) were found 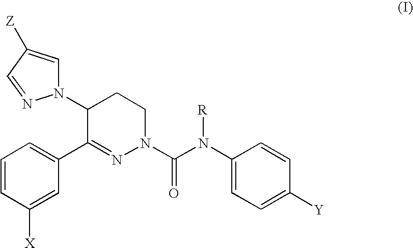
wherein
R represents each optionally substituted alkyl, alkoxycarbonyl, alkenyl, alkynyl, cycloalkyl or cycloalkylalkyl,
X represents cyano, halogen, halogenoalkyl, halogenoalkoxy, aklylthio, alkyl-sulfinyl, alkylsulfonyl, halogenoalkylthio, halogenoalkylsulfinyl or halogeno-alkylsulfonyl,
Y represents cyano, halogen, halogenoalkyl, halogenoalkoxy, halogenoalkylthio, halogenoalkylsulfinyl or halogenoalkylsulfonyl, and
Z represents hydrogen, hydroxy, nitro, cyano, carbamoyl, halogen, alkyl, alkoxy, alkylthio, halogenoalkyl, halogenoalkoxy, halogenoalkylthio, alkoxycarbonyl, alkylaminocarbonyl or dialkylaminocarbonyl

Furthermore, it was found that tetrahydropyridazine derivatives of the formula (I) can be obtained by converting

(a) tetrahydropyridazines of the formula (II) 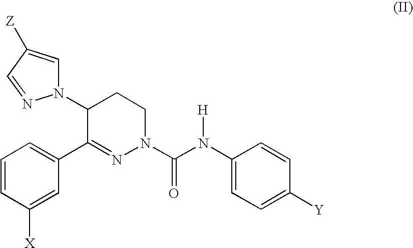
wherein
X, Y and Z have the aforementioned meanings,
with compounds of the formula (III)
   X¹—R (III)
wherein
X¹ represents halogen or the grouping R—O—SO₂—O— and
R has the aforementioned meanings,
optionally in the presence of one or multiple reaction adjuvants and,
optionally, in the presence of one or multiple solvents,
   or

(b) tetrahydropyridazines of the formula (IV) 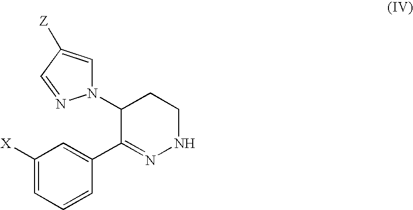
wherein
X and Z have the aforementioned meanings,
with N-substituted arylamines of the formula (V) 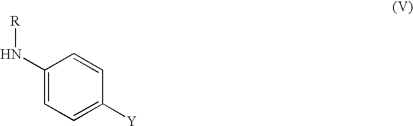
wherein
R and Y have the aforementioned meanings,
in the presence of a reactive carbon dioxide derivative of the formula (VI)
   X²—CO—X² (VI)
wherein
X² and X³ are the same or different and are halogen, alkoxy, halogenoalkoxy, phenoxy or benzyloxy,
optionally in the presence of one or multiple reaction adjuvants and,
optionally, in the presence of one or multiple diluents,
   or

(c) tetrahydropyridazines of the formula (IV) 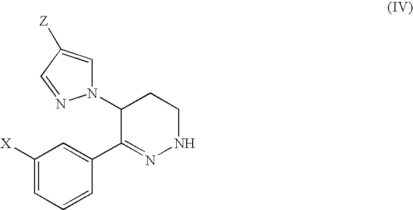
wherein
X and Z have the aforementioned meanings,
with carbamic acid derivatives of the formula (VII) 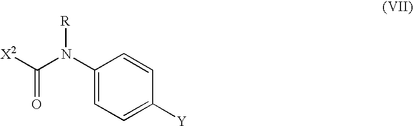
wherein
R, X² and Y have the aforementioned meanings,
optionally in the presence of one or multiple reaction adjuvants and,
optionally, in the presence of one or multiple diluents.

Lastly, it was found that the novel tetrahydropyridazine derivatives of the formula (I) possess very distinctive biological traits and are suitable primarily for controlling animal pests, especially insects, arachnids and nematodes that occur in agriculture, in forests, in stored-product and materials protection, as well as in the sanitation sector.

The tetrahydropyridazine derivatives of the the invention are generally defined by the formula (I).

Preferred substituents or sections of the residues listed in the formulas mentioned above and below are defined as follows:
R represents preferably, any cyano, carboxy, carbamoyl, halogen, C₁-C₄ alkoxy, C₁-C₄ alkylcarbonyl or C₁-C₄ alkoxycarbonyl substituting for alkyl with 1 to 6 carbon atoms; alkoxycarbonyl with up to 6 carbon atoms; either optional cyano or halogen substituting for alkenyl or alkinyl with 2 to 6 carbon atoms each; or each optional cyano, halogen or C₁-C₄ alkyl substituting for cycloalkyl or cycloalkylalkyl with 3 to 6 carbon atoms each in the cycloalkyl group and, optionally, 1 to 4 carbon atoms in the alkyl section.
X represents preferably cyano, halogen, C₁-C₄ halogenoalkyl, C₁-C₄ halogeno-alkoxy, C₁-C₄ alkylthio, C₁-C₄ alkylsulfinyl, C₁-C₄ alkylsulfonyl; C₁-C₄ halogenoalkylthio, C₁-C₄ halogenoalkylsulfinyl or C₁-C₄ halogenoalkylsulfonyl, whereby the halogenoalkyl groups each contain 1 to 5 same or different halogen substituents from the fluorine, chlorine and bromine series.
Y represents preferably cyano, halogen, C₁-C₄ halogenoalkyl, C₁-C₄ halogeno-alkoxy, C₁-C₄ halogenoalkylthio, C₁-C₄ halogenoalkylsulfinyl or C₁-C₄ halogeno-alkylsulfonyl, whereby the halogenoalkyl groups each contain 1 to 5 same or different halogen substituents from the fluorine, chlorine and bromine series.
Z represents preferably hydrogen, hydroxy, nitro, cyano, carbamoyl, halogen, C₁-C₄ alkyl, C₁-C₄ alkoxy, C₁-C₄ alkylthio; C₁-C₄ halogenoalkyl, C₁-C₄ halogenoalkoxy, C₁-C₄ halogenoalkylthio, whereby the halogenoalkyl groups each contain 1 to 5 same or different halogen substituents from the fluorine, chlorine and bromine series; C₁-C₄ alkoxy-carbonyl, C₁-C₄ alkyl-amino-carbonyl or C₁-C₄ dialkyl-amino-carbonyl.
R represents particularly preferably any cyano, carboxy, carbamoyl, fluorine, chlorine, methoxy, ethoxy, n- or i-propoxy, acetyl, propionyl, n- or i-butyroyl, methoxycarbonyl, ethoxycarbonyl, n- or i-propoxycarbonyl substituting for methyl, ethyl, n- or i-propyl, n-, i- or s-butyl; any optional cyano, fluorine and/or chlorine substituting for ethenyl, propenyl, butenyl, ethinyl, propinyl or butinyl; or any optional cyano, fluorine, chlorine, methyl or ethyl substituting for cyclopropyl, cyclobutyl, cyclopentyl, cyclohexyl, cyclopropylmethyl, cyclobutylmethyl, cyclopentylmethyl or cyclohexylmethyl.
X represents particularly preferably cyano, fluorine, chlorine, bromine, difluoromethyl, trifluoromethyl, dichloromethyl, trichloromethyl, chlorodifluoromethyl, fluorodichloromethyl, difluoromethoxy, trifluoromethoxy, chlorodifluoromethoxy, methylthio, methylsulfinyl, methylsulfonyl, trifluoromethylthio, trifluoromethylsulfinyl or trifluoromethylsulfonyl.
Y represents particularly preferably cyano, fluorine, chlorine, bromine, difluoromethyl, trifluoromethyl, dichloromethyl, trichloromethyl, chlorodifluoromethyl, fluorodichloromethyl, difluoromethoxy, trifluoromethoxy, chlorodifluoromethoxy, trifluoromethylthio, trifluoromethylsulfinyl or trifluoromethylsulfonyl.
Z represents particularly preferably hydrogen, cyano, carbamoyl, chlorine, bromine, methyl, methoxy, methylthio, trifluoromethyl, trifluoromethoxy, trifluoromethylthio, methoxycarbonyl, ethoxycarbonyl, methylaminocarbonyl, ethylaminocarbonyl or dimethylaminocarbonyl.
R represents especially preferably any optional cyano, fluorine, chlorine, methoxy, ethoxy, acetyl, propionyl, methoxycarbonyl, ethoxycarbonyl, n- or i-propoxycarbonyl substituting for methyl, ethyl, n- or i-propyl; any optional cyano, fluorine and/or chlorine substituting for propenyl, butenyl, ethinyl, propinyl or butinyl; or any optional fluorine, chlorine or methyl substituting for cyclopropyl, cyclobutyl, cyclopentyl, cyclohexyl, cyclopropylmethyl, cyclobutylmethyl, cyclopentylmethyl or cyclohexylmethyl.
X represents especially preferably cyano, fluorine, chlorine, bromine, trifluoromethyl, difluoromethoxy, trifluoromethoxy, chlorodifluormethoxy, methylthio, methylsulfinyl, methylsulfonyl, trifluoromethylthio, trifluoromethylsulfinyl or trifluoromethylsulfonyl.
Y represents especially preferably cyano, fluorine, chlorine, bromine, trifluoromethyl, difluoromethoxy, trifluoromethoxy, chlorodifluormethoxy, trifluoromethylthio, trifluoromethylsulfinyl or trifluoromethylsulfonyl.
Z represents especially preferably hydrogen, cyano, carbamoyl, chlorine, bromine, methyl, methoxy, methylthio, trifluoromethyl, trifluoromethoxy or trifluoromethylthio.

Additionally preferred are tetrahydropyridazine derivatives of the formula (I) wherein

R represents cyanomethyl or cyanoethyl; particularly preferably R represents cyanomethyl.

Additionally preferred are tetrahydropyridazine derivatives of the formula (I), wherein R represents methoxycarbonyl.

Additionally preferred are tetrahydropyridazine derivatives of the formula (I), wherein R represents methyl or ethyl.

Additionally preferred are tetrahydropyridazine derivatives of the formula (I), wherein R represents methoxymethyl.

Additionally preferred are tetrahydropyridazine derivatives of the formula (I), wherein X represents cyano.

Additionally preferred are tetrahydropyridazine derivatives of the formula (I), wherein X represents trifluoromethyl.

Additionally preferred are tetrahydropyridazine derivatives of the formula (I) wherein X represents halogen; particularly preferably X represents fluorine, chlorine or bromine.

Additionally preferred are tetrahydropyridazine derivatives of the formula (I), wherein R represents cyanomethyl and X represents trifluoromethyl.

Additionally preferred are tetrahydropyridazine derivatives of the formula (I), wherein Z represents cyano.

Additionally preferred are tetrahydropyridazine derivatives of the formula (I) in which Z represents halogen; particularly preferably Z represents chlorine or bromine, especially preferably Z represents chlorine.

The definitions of residues listed above or in the preferred and definitions sections apply to the end products and to the starting and intermediate products, accordingly. These defined residues can be combined as desired among themselves, that is, also among the respective preferred sections.

According to the invention, compounds of the formula (I) which comprise a combination of the meanings listed above as preferred, are preferred.

According to the invention, compounds of the formula (I) that comprise a combination of the meanings listed above as particularly preferred, are particularly preferred.

According to the invention, compounds of the formula (I) that comprise a combination of the meanings listed above as especially preferred, are especially preferred.

In the above and following definitions of residues listed, hydrocarbon residues, such as alkyl—including when combined with heteroatoms such as alkoxy—are wherever possible either straight-chained or branched.

For example, if 4-(4-chloropyrazole-1-yl)-3-(3-fluorophenyl)-1-(4-trifluoromethyl-phenyl-amino-carbonyl-1,4,5,6-tetrahydro-pyridazine and chloromethylcyclopropane are used as starting materials, the reaction using the process of the invention (a) can be rendered by the following formula diagram:

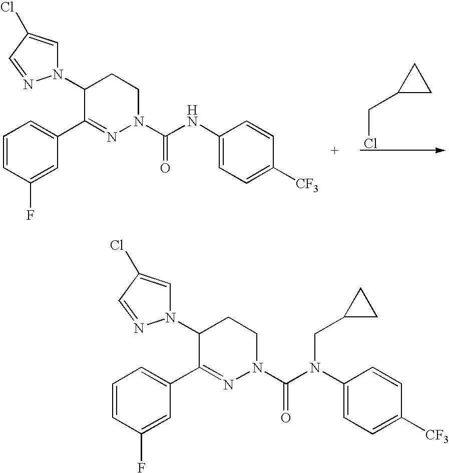

For example, if 4-(4-cyanopyrazole-1-yl)-3-(3-difluoromethoxy-phenyl)-1,4,5,6-tetrahydropyridazine, N-(2-cyanoethyl)-4-bromine-aniline and phosgene are used as starting materials, the reaction using the process of the invention (b) can be rendered by the following formula diagram:

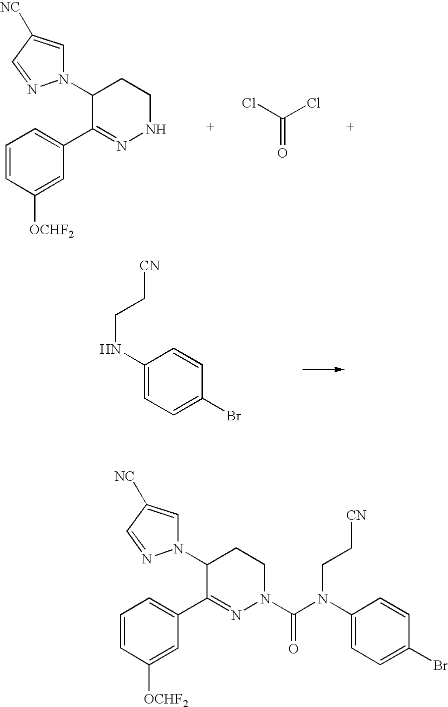

For example, if 4-(4-cyanopyrazole-1-yl)-3-(3-trifluoromethoxy-phenyl)-1,4,5,6-tetrahydropyridazine and 4-bromophenyl(cyanomethyl)carbamine chloride are used as starting materials, the reaction using the process of the invention (c) can be rendered by the following formula diagram:

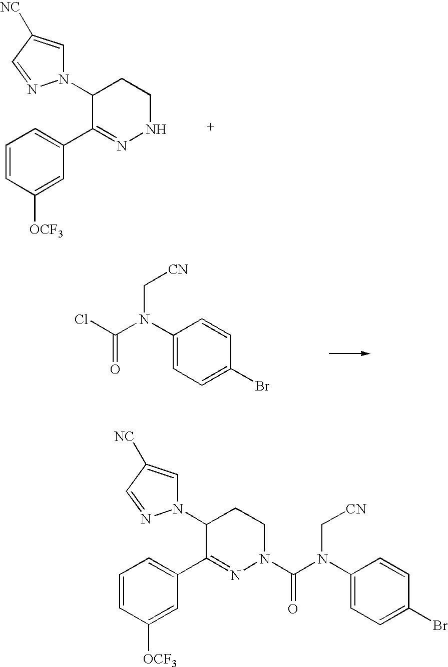

The tetrahydropyridazines to be used as starting materials for the process of the invention (a) are generally defined by the formula (II). The tetrahydropyridazines of the formula (II) are the object of DE-A 101 36 066.

The tetrahydropyridazines of the formula (II) can be obtained, for example, by converting

(d) tetrahydropyridazines of the formula (IV) 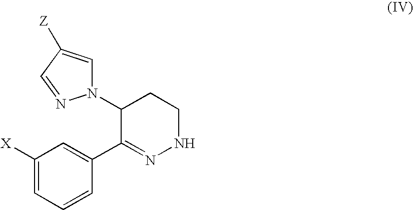
wherein
X and Z have the aforementioned meanings,
with isocyanates of the formula (VIII) 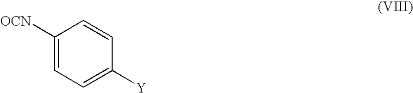
wherein
Y has the aforementioned meanings,
optionally, in the presence of a diluent at temperatures between 0° C. and 100° C. (cf. the preparation examples).

The process of the invention (d) is preferably carried out using diluents. Practically all inert organic solvents can be considered diluents. Included are preferably aliphatic and aromatic—optionally—halogenated hydrocarbons such as pentane, hexane, heptane, cyclohexane, petrolether, benzin, ligroine, benzene, toluol, xylol, methylene chloride, ethylene chloride, chloroform, tetrachlorocarbon, chlorobenzene and o-dichlorobenzene; ethers such as diethyl- and dibutilether, glycoldimethylether and diglycoldimethylether, tetrahydrofuran and dioxan; ketones such as acetone, methyl-ethyl-, methyl-isopropyl- or methyl-isobutyl-ketone; esters such as methyl acetate or ethyl acetate; nitriles such as acetonitrile o r propionitrile; amides such as dimethylformamide, dimethylacetamide and N-methylpyrrolidone; as well as dimethylsulfoxide, tetramethylenesulfon or hexamethylenephosphoric acid triamide.

The reaction temperatures can be varied widely for the process of the invention (d). As a rule, use temperatures between 0° C. and 100° C., preferably temperatures between 10° C. and 80° C.

The, compounds of the formula (m) to be additionally used as starting materials for the process of the invention (a) are known organic synthesis-chemicals. If X¹ represents halogen, this means the preferred chemicals are chlorine, bromine and iodine.

The tetrahydropyridazines to be used as starting materials for the processes of the invention (b) and (c) are generally defined by the formula (IV). The tetrahydropyridazines of the formula (IV) are the object of DE-A 101 36 066.

The tetrahydropyridazines of the formula (IV) can be obtained in the generally customary manner, by converting

(e) ω-chlorinated ketones of the formula (IX) 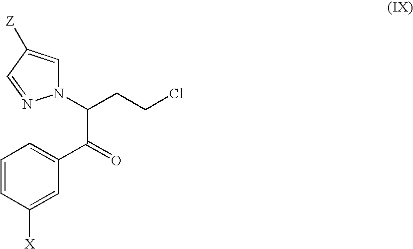
wherein
X and Z have the aforementioned meanings,
with hydrazine hydrate (NH₂—NH₂×H₂O), optionally, in the presence of a diluent, such as ethanol, at temperatures between 0° C. and 50° C. (cf. the preparation examples).

The ω-chlorinated ketones of the formula (IX) are also object of DE-A 101 36 066. They can be obtained in the generally customary manner, by converting

(f) dihalogenated ketones of the formula (X)) 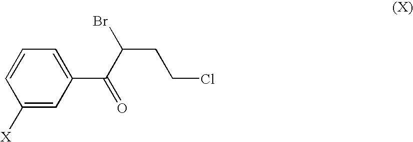
wherein
X has the aforementioned meanings,
with known pyrazoles of the formula (XI) 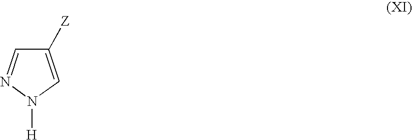
wherein
Z has the aforementioned meanings,
preferably in the form of hydrogen halogenides (e.g., hydrochloride), optionally in the presence of an inert diluent (e.g., acetonitrile, acetone or N,N-dimethylformamide) and, optionally, in the presence of bases (e.g., potassium or calcium carbonate; sodium or potassium-t-butylate; sodium, potassium or calcium hydride; triethylamine or ethyl diisopropylarnine), at temperatures of −20° C. to +40° C. (cf. also the preparation examples).

The N-substituted arylamines of the formula (V) to be additionally used as starting materials for process of the invention (b) are known organic synthesis-chemicals.

The carbonic acid derivatives of the formula (VI) to be additionally used as starting materials for process of the invention (b) are known synthesis-chemicals. In the formula (VI) X² and X³ are preferably chlorine or trichloromethoxy. Examples of the compounds for the formula (VI) include phosgene, diphosgene and triphosgene.

The carbamic acid derivatives of the formula (VII) to be additionally used as starting materials for the process of invention (c) are known and/or can be manufactured using known processes (cf. DE-A 24 29 523, U.S. Pat. No. 3,991,071, U.S. Pat. No. 4,154,945, DE-A 25 36 192, U.S. Pat. No. 3,925,397, U.S. Pat. No. 4,033,972). In the formula (VI) R and Y have as preferred, particularly preferred or especially preferred those meanings that are given in the above definitions of the respective groups for the compounds of the formula (I) as preferred, particularly preferred or especially preferred; x² represents preferably halogen, particularly preferably chlorine.

The processes (a), (b) and (c) of the invention for the preparation of the compounds of the formula (I) are performed preferably with the use of one or multiple reaction adjuvants. The customary inorganic or organic bases or acid acceptors are considered reaction adjuvants. Included preferably are alkali-metal or alkaline-earth-metal acetates, amides, carbonates, hydrogencarbonates, hydrides, hydroxides or alkanolates, such as sodium, potassium or calcium-acetate; lithium-, sodium-, potassium- or calcium-amide; sodium-, potassium- or calcium-carbonate; sodium-, potassium- or calcium-hydrogencarbonate; lithium-, sodium-, potassium- or calcium-hydride; lithium-, sodium-, potassium- or calcium-hydroxide; sodium- or potassium- -methanolate -ethanolate, -n- or -i-propanolate, -n-, -i-, -s- or -t-butanolate; additionally also basic organic nitrogen compounds, such as trimethylamine, triethylamine, tripropylamine, tributylamine, ethyl-diisopropylamine, N,N-dimethyl-cyclohexylamine, dicyclohexylamine, ethyl-dicyclohexylamine, N,N-di-methyl-aniline, N,N-dimethyl-benzylamine, pyridine, 2-methyl-, 3-methyl-, 4-methyl-, 2,4-dimethyl-, 2,6-dimethyl-, 3,4-dimethyl- and 3,5-dimethyl-pyridine, 5-ethyl-2-methyl-pyridine, 4-dimethylamino-pyridine, N-methyl-piperidine, 1,4-diazabicyclo[2,2,2]octane (DABCO), 1,5 diazabicyclo[4,3,0]non-5-ene (DBN), or 1,8 diazabicyclo[5,4,0]-7-undecene (DBU).

The processes (a), (b) and (c) of the invention are performed preferably using one or multiple diluents. Practically all inert organic solvents can be considered diluents. Included preferably are aliphatic and aromatic, optionally, halogenated hydrocarbons such as pentane, hexane, heptane, cyclohexane, petrolether, benzine, ligroine, benzene, toluol, xylol, methylene chloride, ethylene chloride, chloroform, tetrachlorocarbon, chlorobenzene and o-dichlorobenzene; ethers such as diethyl- and dibutylether, glycoldimehtylether and diglycoldimethylether, tetrahydrofuran and dioxan; ketones such as acetone, methylethyl, methyl isopropyl or methyl isobutyl ketone; esters such as methyl acetate or ethyl acetate esters; nitrites such as acetonitrile or propionitrile; amides such as dimethylformamide, dimethylacetamide and N-methyl pyrrolidone; as well as dimethylsulfoxide, tetramethylenesulfon and hexamethylene phosphoric acid triamide.

The reaction temperatures can be varied within a wide range for processes (a), (b) and (c) of the invention. In general, temperatures between 0° C. and 100° C. are used, preferably temperatures between 10° C. and 80° C.

The processes of the invention are generally carried out under normal pressure. However, it is also possible to work under higher or lower pressure conditions.

In general, the respective starting materials required for executing the processes of the invention are employed in approximately equimolar amounts. However, it is also possible to use one of the respectively included components in greater excess. For the processes of the invention, preparations are done in accordance with customary methods (cf. the preparation examples).

The active ingredients, having good plant tolerability, favorable toxicity to warm-blooded animals, and good environmental tolerability, are suitable for protecting plants and plant organs, increasing harvests, improving the quality of the harvest and controlling animal pests, especially insects, arachnids and nematodes that occur in agriculture, forests, gardens and recreational facilities, in stored product and materials protection, as well as in the sanitation sector. They can be used preferably as pesticides. They are effective against normally sensitive and resistant species, as well as against all developmental stages, or individual stages. The aforementioned pests include:

From the order Isopoda, e.g., Oniscus asellus, Armadillidium vulgare, Porcellio scaber.

From the order Diplopoda, e.g., Blaniulus guttulatus.

From the order Chilopoda, e.g., Geophilus carpophagus, Scutigera spp.

From the order Symphyla, e.g., Scutigerella immaculata.

From the order Thysanura, e.g., Lepisma saccharina.

From the order Collembola, e.g., Onychiurus armatus.

From the order Orthoptera, e.g., Acheta domesticus, Gryllotalpa spp., Locusta migratoria migratorioides, Melanoplus spp., Schistocerca gregaria.

From the order Blattaria, e.g., Blatta orientalis, Periplaneta americana, Leucophaea maderae, Blattella germanica.

From the order Dermaptera, e.g., Forficula auricularia.

From the order Isoptera, e.g., Reticulitermes spp.

From the order Phthiraptera, e.g., Pediculus humanus corporis, Haematopinus spp., Linognathus spp., Trichodectes spp., Damalinia spp.

From the order Thysanoptera, e.g., Hercinothrips femoralis, Thrips tabaci, Thrips palmi, Frankliniella occidentalis.

From the order Heteroptera, e.g., Eurygaster spp., Dysdercus intermedius, Piesma quadrata, Cimex lectularius, Rhodnius prolixus, Triatoma spp.

From the order Homoptera, e.g., Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp., Psylla spp.

From the order Lepidoptera e.g, Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp., Oulema oryzae.

From the order Coleoptera, e.g., Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica, Lissorhoptrus oryzophilus.

From the order Hymenoptera, e.g., Diprion spp., Hoplocampa spp., Lasius spp., Monomorium pharaonis, Vespa spp.

From the order Diptera, e.g., Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp., Liriomyza spp.

From the order Siphonaptera, e.g., Xenopsylla cheopis, Ceratophyllus spp.

From the class Arachnida, e.g., Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp., Brevipalpus spp.

The plant-parasitic nematodes include, e.g., Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp., Bursaphelenchus spp.

The compounds of the invention of the formula (I) are characterized in particular by having a potent efficacy against butterfly larvae, such as Heliothis armigera, Plutella xylostella, Spodoptera exigua and Spodoptera frugiperda, and against bettle larvae, such as Phaedon cochleariae.

The compounds of the invention can also be optionally used in certain concentrations or application amounts as herbicides and microbicides, for example as fungicides, antimycotics and bactericides. They can also be employed optionally as precursors or intermediates for the synthesis of additional active ingredients.

In accordance with the invention all plants and plant parts can be treated. Plants are understood here to be all plants and plant populations, such as desirable and undesirable wild plants or cultivated plants (including naturally occurring cultivated plants). Cultivated plants may be plants that can be obtained through conventional breeding and optimization methods or through biotechnological and genetic engineering methods or combinations of these methods, including transgenic plants and including plant varieties that can and cannot be protected by plant breeder's rights. Plant parts are understood to be all above-ground and underground plant parts and organs, such as the sprout, leaf, flower and root, whereby leaves, needles, stalks, stems, flowers, receptacles, fruits and seeds, as well as roots, tubers, rhizomes are listed as examples. Plant parts also include harvested crops, as well as vegetative and generative propagation material, for example shoots, tubers, rhizomes, runners and seeds.

The treatment of plants and plant parts with the active ingredients in accordance with the invention occurs directly, or through action on the environment, habitat or storage space in accordance with customary treatment methods, e.g. dipping, spraying, vaporizing, nebulizing, sprinkling, coating, injection, and, with propagation material, in particular seeds, by one-layer or multi-layer enclosing of the seeds.

The active ingredients can be transformed into the usual formulations, such as solutions, emulsions, wettable powders, suspensions, powders, dusts, pastes, soluble powders, granulates, suspension emulsion concentrates, natural and synthetic substances impregnated with the active ingredient and microencapsulations in polymer substances.

These formulations are produced in known manners, e.g., by mixing the active ingredients with extenders, i.e., liquid solvents and/or solid carriers, optionally using surfactants, i.e., emulsifiers and/or dispersants and/or foaming agents.

In the case of water being used as an extender, organic solvents, for example, can also be used as auxiliary solvents. The primary liquid solvents to be considered are: Aromatics, such as xylol, toluol or alkylnaphthalenes; chlorinated aromatics and chlorinated aliphatic hydrocarbons, such as chlorobenzenes, chloroethylenes, and methylene chloride; aliphatic hydrocarbons such as cyclohexane and paraffins, e.g., crude oil fractions, mineral and plant oils; alcohols, such as butanol and glycol, as well as their ethers and esters; ketones such as acetone, methyl ethyl ketone, methyl isobutyl ketones, and cyclohexane; highly polarized solvents, such as dimethyl formamide and dimethyl sulfoxide; and water.

Solid carriers to be considered are:

e.g., ammonium salts and natural crushed rock, such as kaolins, aluminas, talc, chalk, silica, attapulgite, montmorillonite and diatomaceous earth; and synthetic crushed rock, such as highly disperse silicic acid, aluminium oxide and silicates; solid carriers for granulates to be considered are: e.g., crushed and fractionated natural stone such as calcite, marble, pumice, sepiolite, dolomite, as well as synthetic granulates from inorganic and organic rock flours, and granulates from organic material such as sawdust, coconut shells, corn cobs and tobacco stalks; emulsifiers and/or foaming agents to be considered are: e.g., non-ionogenic and anionic emulsifiers, such as polyoxyethylene fatty acid esters, polyoxyethylene fatty alcohol ethers, e.g. alkylaryl polyglycolether, alkylsulfonates, alkylsulfates, arylsulfonates as well as protein hydrolysates; dispersants to be considered are: e.g., lignosulfonate waste liquor and methyl cellulose.

In the formulations, deposit builders such as carboxymethylcellulose, natural and synthetic powdered, granulated or latex-shaped polymers may be used, such as gum arabic, polyvinyl alcohol, polyvinyl acetate, as well as natural phospholipids such as cephalines and lecithins and synthetic phospholipids. Other additives may include mineral and vegetable oils.

Colorants such as inorganic pigments, e.g., iron oxide, titanium oxide, ferrocyanide blue and organic colorants, such as alizarin-, azo- and metalphthalocyanine dyes and trace nutrients such as iron salts, manganese, boron, copper, cobalt, molybdenum and zinc can be used.

The formulations generally comprise between 0.1 and 95% w/w of the active ingredient, preferably between 0.5% and 90%.

The active ingredients according to the invention can exist in customary formulations, as well as in the application forms prepared from these formulations in mixture with other active ingredients, such as insecticides, attractants, sterilants, bactericides, acaricides, nematicides, fungicides, growth-regulating agents and herbicides. Insecticides include, for example, phosphoric acid esters, carbamates, carbonic acid esters, chlorinated hydrocarbons, phenylureas, and substances produced by microorganisms, and the like.

The following are examples of particularly advantageous mixing partners:

Fungicides:

2-phenylphenol; 8-hydroxyquinoline sulfate; acibenzolar-S-methyl; aldimorph; amido-flumet; ampropylfos; ampropylfos potassium; andoprim; anilazine; azaconazole; azoxystrobin; benalaxyl; benodanil; benomyl; benthiavalicarb isopropyl; benzamacril; benzamacril isobutyl; bilanafos; binapacryl; biphenyl; bitertanol; blasticidin-S; bromuconazole; bupirimate; buthiobate; butylamine; calcium polysulfide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofluram; dagger G; debacarb; dichlofluanide; dichlone; dichlorophen; diclocymet; diclomezine; dicloran; diethofencarb; difenoconazole; diflunmetorim; dimethirimol; dimethomorph; dimoxystrobin; diniconazole; diniconazole-m; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexarnid; fenitropan; fenoxanil; fenpiclonil; fenpropidin; fenpropimorph; ferbam; fluazinam; flubenzimine; fludioxonil; flumetover, flumorph; fluorornide; fluoxastrobin; fluquinconazole; flurprimidol; flusilazole; flusulfamide; flutolanil; flutriafol; folpet; fosetyl-al; fosetyl-sodium; fuiberidazole; furalaxyl; furaetpyr; fuircarbanil; furmecyclox; guazatine; hexachlorobenzene; hexaconazole; hymexazol; imazalil; imibenconazole; iminoctadine triacetate; iminoctadine tris(albesilate); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazol; pefurazoate; penconazole; pencycuron; phosdiphen; phthalide; picoxystrobin; piperalin; polyoxins; polyoxorim; probenazole; prochloraz; procymidone; propamocarb; propanosine sodium; propiconazole; propineb; proquinazid; prothioconazole; pyraclostrobin; pyrazophos; pyrifenox; pyrimethanil; pyroquilon; pyroxyfur, pyrrolnitrine; quinconazole; quinoxyfen; quintozene; simeconazole; spiroxamine; sulfur; tebuconazole; tecloftalam; tecnazene; tetcyclacis; tetraconazole; thiabendazole; thicyofen; thifluzamide; thiophanate methyl; thiram; tioxymid; tolclofos methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)-N-[2-[4-[[3-(4-chlorophenyl)-2-propinyl]oxy]-3-methoxyphenyl]ethyl]-3-methyl-2-[(methylsulfonyl)amino]-butanamide; 1-(1-naphthalenyl)-1H-pyrrol-2,5-dione; 2,3,5,6-tetrachloro-4-(methylsulfonyl)-pyridine; 2-amino-4-methyl-N-phenyl-5-thiazol-carboxamide; 2-chloro-N-(2,3-dihydro-1,1,3-trimethyl-1H-inden-4-yl)-3-pyridine carboxamide; 3,4,5-trichloro-2,6-pyridine dicarbonitril; actinovate; cis-1-(4-chlorophenyl)-2-(1H-1,2,4-triazol-1-yl)-cycloheptanol; methyl-(2,3-dihydro-2,2-dimethyl-1H-inden-1-yl)-1H-imidazol-5-carboxylate; monopotassiumcarbonate; N-(6-methoxy-3-pyridinyl)-cyclopropane carboxamide; N-butyl-8-(1,1-dimethylethy)-1-oxaspiro[4.5]decan-3-amine; sodium tetrathiocarbonate;

as well as copper salts and preparations, such as Bordeaux mixture; copper hydroxide; copper naphthenate; copper oxychloride; copper sulfate; cufraneb; cuprous oxide; mancopper; oxine copper.

Bactericides:

bronopol, dichlorophen, nitrapyrin, nickel dimethylidithiocarbamate, kasugamycin, octhilinon, furan-carboxylic acid, oxytetracycline, probenazole, streptomycin, tecloftalam, copper sulfate and other copper preparations.

Insecticides/Acaricides/Nematicides:

abamectin, ABG-9008, acephate, acequinocyl, acetamiprid, acetoprole, acrinathrin, AKD-1022, AKD-3059, AKD-3088, alanycarb, aldicarb, aldoxycarb, allethrin, allethrin irisomers, alpha cypermethrin (alphamethrin), amidoflumet, aminocarb, amitraz, avermectin, AZ-60541, azadirachtin, azamethiphos, azinphos methyl, azinphos ethyl, azocyclotin, Bacillus popilliae, Bacillus sphaericus, Bacillus subtilis, Bacillus thuringiensis, Bacillus thuringiensis strain EG-2348, Bacillus thuringiensis strain GC-91, Bacillus thuringiensis strain NCTC-11821, baculoviruses, Beauveria bassiana, Beauveria tenella, bendiocarb, benfuracarb, bensultap, benzoximate, beta-cyfluthrin, beta-cypermethrin, bifenazate, bifenthrin, binapacryl, bioallethrin, bioallethrin-S-cyclopentyl-isomer, bioethanomethrin, biopermethrin, bioresmethirn, bistrifluron, BPMC, brofenprox, bromophos ethyl, bromopropylate, bromfenvinfos (methyl), BTG-504, BTG-505, bufencarb, buprofezin, butathiofos, butocarboxim, butoxycarboxim, butylpyridaben, cadusafos, camphechlor, carbaryl, carbofuran, carbophenothion, carbosulfan, cartap, CGA-50439, chinomethionat, chlordane, chlordimeform, chloethocarb, chlorethoxyfos, chlorfenapyr, chlorfenvinphos, chlorfluazuron, chlormephos, chlorobenzilate, chloropicrin, chlorproxyfen, chlorpyrifosmethyl, chlorpyrifos (ethyl), chlovaporthrin, chromafenozide, cis-cypermethrin, cis-resmethrin, cis-pemedirin, clocythrin, cloethocarb, clofentezine, clothianidin, clothiazoben, codlemone, coumaphos, cyanofenphos, cyanophos, cycloprene, cycloprothiin, cydia pomonella, cyfluthrin, cyhalothrin, cyhexatin, cypermedrin, cyphenothrin (1R-trans-isomer), cyromazine, DDT, deltamethrin, demeton-S-methyl, demeton-S-methylsulphon, diafenthiuron, dialifos, diazinon, dichlofenthion, dichlorvos, dicofol, dicrotophos, dicyclanil, diflubenzuron, dimethoate, dimethylvinphos, dinobuton, dinocap, dinetofwran, diofenolan, disulfoton, docusat-sodium, dofenapyn, DOWCO-439, eflusilanate, emamectin, emamectin-benzoate, empenthrin (IR-isomer), endosulfan, Entomopthora spp., EPN, esfenvalerate, ethiofencarb, ethiprole, ethion, ethoprophos, etofenprox, etoxazole, etrimfos, famphur, fenamiphos, fenazaquin, fenbutatin oxide, fenfluthrin, fenitrothion, fenobucarb, fenothiocarb, fenoxacrim, fenoxycarb, fenpropathrin, fenpyrad, fenpyrithrin, fenpyroximate, fensulfothion, fenthion, fentrifanil, fenvalerate, fipronil, flonicamid, fluacrypyrim, fluazuron, flubenzimine, flubrocythrinate, flucycloxuron, flucythrinate, flufenerim, flufenoxuron, flufenprox, flumeffiin, flupyrazofos, flutenzin (flufenzine), fluvalinate, fonofos, formetanate, formothion, fosmethilan, fosthiazate, fubfenprox (fluproxyfen), furathiocarb, gamma-HCH, gossyplure, grandlure, granuloseviren, halfenprox, halofenozide, HCH, HCN-801, heptenophos, hexaflumuron, hexythiazox, hydramethylnone, hydroprene, IKA-2002, imidacloprid, imiprothrin, indoxacarb, iodofenphos, iprobenfos, isazofos, isofenphos, isoprocarb, isoxathion, ivermectin, japonilure, kadethrin, nuclear polyhedrosis viruses, kinoprene, lambda cyhalothrin, lindane, lufenuron, malathion, mecarbam, mesulfenfos, metaldehyd, metam-sodium, methacrifos, methamidophos, metharhizium anisopliae, metharhizium flavoviride, methidathion, methiocarb, methomyl, methoprene, methoxychlor, methoxyfenozide, metolcarb, metoxadiazone, mevinphos, milbemectin, milbemycin, MKI-245, MON45700, monocrotophos, moxidectin, MTI-800, naled, NC-104, NC-170, NC-184, NC-194, NC-196, niclosamnide, nicotine, nitenpyram, nithiazine, NNI-0001, NNI-0101, NNI-0250, NNI-9768, novaluron, noviflumuron, OK-5101, OK-5201, OK-9601, OK-9602, OK-9701, OK-9802, omethoate, oxamyl, oxydemeton methyl, paecilomyces fumosoroseus, parathion methyl, parathion (ethyl), permethrin (cis-, trans-), petroleum, PH-6045, phenothiin (1R-trans isomer), phenthoate, phorate, phosalone, phosmet, phosphamidon, phosphocarb, phoxim, piperonyl butoxide, pirimicarb, pirimiphos methyl, pirimiphos ethyl, prallethrin, profenofos, promecarb, propaphos, propargite, propetamphos, propoxur, prothiofos, prothoate, protrifenbute, pymetrozine, pyraclofos, pyresmethrin, pyrethrum, pyridaben, pyridalyl, pyridaphenthion, pyridathion, pyrimidifen, pyriproxyfen, quinalphos, resmethrin, RH-5849, ribavirin, RU-12457, RU-15525, S-421, S-1833, salithion, sebufos, SI-0009, silafluofen, spinosad, spirodiclofen, spiromesifen, sulfluramid, sulfotep, sulprofos, SZI-121, tau-fluvalinate, tebufenozide, tebufenpyrad, tebupirimfos, teflubenzuron, tefluthrin, temephos, temivinphos, terbam, terbufos, tetrachlorvinphos, tetradifon, tetrametlrin, tetrametinn (IR-isomer), tetrasul, theta-cypermethrin, thiacloprid, thiamethoxam, thiapronil, thiatriphos, thiocyclam hydrogen oxalate, thiodicarb, thiofanox, thiometon, thio sultap-sodium, thuringiensin, tolfenpyrad, tralocythrin, tralomethrin, transfluthrin, triarathene, triazamate, triazophos, triazuron, trichlophenidine, trichlorfon, triflumuron, trimethacarb,

vamidothion, vaniliprole, verbutin, Verticillium lecanii, WL-108477, WL-40027, YI-5201, YI-5301, YI-5302, XMC, Xylylcarb, ZA-3274, zeta-cypermethrin, zolaprofos, ZXI-8901, the compound 3-methyl phenyl propylcarbamate (tsumacide Z), the compound 3-(5-chloro-3-pyridinyl)-8-(2,2,2-trifluoroethyl)-8-azabicyclo[3.2.1]octane-3-carbonitril (CAS Reg. No. 185982-80-3) and the corresponding 3-endo-isomers (CAS Reg. No. 185984-60-5) (cf WO-96/37494, WO-98/25923),

as well as preparations that contain plant extracts that have an insecticidal effect, nematodes, fungi or viruses.

A mixture with other known active ingredients, such as herbicides, fertilizers, growth regulators, safener or semiochemicals is also possible.

The active ingredients according to the invention can furthermore exist in use as insecticides in their customary formulations, as well as in the application forms prepared from these formulations in mixture with synergistic agents. Synergistic agents are compounds that enhance the effect of the active ingredients without the synergistic agent necessarily being active itself.

The active ingredients according to the invention can furthermore exist in use as insecticides in their customary formulations, as well as in the application forms prepared from these formulations in mixture with inhibiting agents that reduce the breakdown of the active ingredient after application in the area around the plant, on the surface of the plant parts or in plant tissues.

The active ingredient content of the application forms prepared from the customary formulations can vary widely. The active ingredient concentration of the application forms can range from 0.0000001 to 95% w/w of the active ingredient, preferably between 0.0001 and 1% w/w.

The application occurs in a customary manner appropriate for the application forms.

For application to sanitation and stored-product pests the active ingredients are characterized by an outstanding residual effect on wood and clay, as well as with good alkali stability on limed substrates.

As already mentioned above, according to the invention all plants and parts thereof can be treated. In a preferred embodiment plant species and plant varieties and their parts that grow in the wild or that are cultivated using conventional biological breeding methods, such as cross-breeding or protoplast fusion, are treated. In another preferred embodiment transgenic plants and plant varieties and their parts that were obtained using genetic engineering (GMOs), possibly in combination with conventional methods, are treated. The terms “parts” and “parts of plants” and “plant parts” were defined above.

According to the invention, the treatment of both commercially obtainable plant varieties and plant varieties in use is particularly preferred. Plant varieties are understood to be plants with new traits that are bred using conventional breeding methods, mutagenesis or using recombinant DNA techniques. These can be varieties, biotypes and genotypes.

Depending on the plant species or variety, its location and growth conditions (soil, climate, vegetation period, nutrients), superadditive (synergistic) effects can also occur when treated in accordance with the invention. So, for example, reduced application amounts and/or expansion of the spectrum of action and/or an improvement of the action of the materials and agents of the invention, better plant growth; increased tolerance of higher and lower temperatures; increased tolerance of drought, or water or salt content in the soil; increased flowering capability, easier harvesting; faster maturation; increased harvest yields; higher quality and/or increased nutritional value of the harvested product; improved ability to be stored and/or for the harvested product to be processed, which are above and beyond the actually anticipated effects, are possible.

According to the invention, preferred transgenic (genetically engineered) plants or plant varieties to be treated include all plants that acquired genetic material that imparts particularly advantageous and valuable traits to these plants by way of genetic modification. Examples of such traits are better plant growth, increased tolerance of high and low temperatures; increased tolerance of drought or water or salt content in the soil; increased flowering capability; easier harvesting; faster maturation; increased harvest yields; higher quality and/or increased nutritional value of the harvested product; improved ability to be stored and/or for the harvested product to be processed. Additional and particularly important examples of such traits are an increased defense of the plants against animal and microbial pests, such as insects, mites, plant-pathogenic fungi, bacterias and/or viruses, as well as an increased tolerance of plants to certain herbicidal active ingredients. Examples of transgenic plants include the important cultivated plants, such as grains (wheat, rice), corn, soy, potatoes, cotton, tobacco, rapeseed, and fruiting plants (including the fruits: apple, pear, citrus fruit and grape), whereby corn, soy, potatoes, cotton, tobacco and rapeseed are of particular note. Traits of particular note include increased plant defenses against insects, arachnids, nematodes and snails through toxins that are created in the plant, in particular those that are created by genetic material from Bacillus Thuringiensis (e.g., from the genes CryIA(a), CryIA(b), CryIA(c), CryIIA, CryIIIA, CryIIIB2, Cry9c Cry2Ab, Cry3Bb and CryIF, and their combinations) (referred to below as “Bt plants”). Other traits of particular note include the increased defense of plants agains fungi, bacteria, and viruses through systemically acquired resistance (SAR), systemine, phytoalexines, elicitors and resistant genes and correspondingly expressed proteins and toxins. Further traits of particular note include the increased tolerance of the plants to certain herbicidal active ingredients, for example imidazolinones, sulfonylurea agents, glyphosates and phosphinotricin (e.g., “PAT” gene). The genes that impart the respectively desired traits can also occur in combinations with one another in the transgenic plant. Examples of GMO plants include varieties of corn, cotton, soy and potatoes that are sold under the trade names YIELD GARD® (e.g., corn, cotton, soy), KnockOut® (e.g., corn), StarLink® (e.g., corn), Bollgard® (cotton), Nucotn® (cotton) and NewLeaf® (potatoes). Examples of herbicide-tolerant plants include varieties of corn, cotton and soy that are sold under the trade names Roundup Ready® (tolerant of glyphosates, eg., corn, cotton, soy), Liberty Link® (tolerant of phosphinotricin, e.g., rapeseed), EMI® (tolerant of imidazolinones) and STS® (tolerant of sulfonylureas, e.g., corn). Herbicide resistant plants (bred conventionally for herbicide tolerance) also incude varieties sold under the trade name Clearfield® (e.g., corn). Naturally these statements also apply to plant varieties that will be developed in the future or that will come on the market in the future, having these traits or genetic traits deveoloped in the future.

According to the invention, the listed plants can be treated particularly advantageously with the compounds of the general formula (I) or with the active ingredient mixtures of the invention. The preferred areas listed above for the active ingredients or mixtures also apply to the treatment of these plants. Of particular note is the treatment of plants with the specially outlined compounds and mixtures listed in the aforementioned text.

The active ingredients according to the invention act not only against plant, sanitation and stored-product pests, but also in the veterinary medicine sector against animal parasites (ectoparasites) such as hard ticks, soft ticks, mange mites, leaf mites, flies (biting and licking), parasitic fly larvae, lice, hair lice, feather lice and fleas. These parasites include:

From the order Anoplura, e.g., Pediculus humanus corporis, Haematopinus spp., Linognathus spp., Trichodectes spp., Damalinia spp.

From the order Mallophaga and the subclasses Amblycera and Ischnocera e.g., Trimenopon spp., Menopon spp., Trinoton spp., Bovicola spp., Werneckiella spp., Lepikentron spp., Damalina spp., Trichodectes spp., Felicola spp.

From the order Diptera and the subclasses Nematocera and Brachycera e.g., Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp.

From the order Siphonaptera e.g., Pulex spp., Ctenocephalides spp., Xenopsylla spp., Ceratophyllus spp.

From the order Heteroptera e.g., Cimex spp., Triatoma spp., Rhodnius spp., Panstrongylus spp.

From the order Blattaria, e.g., Blatta orientalis, Periplaneta americana, Blattella germanica, Supella spp.

From the subclass Acari (Acarina) and the orders Meta- and Mesostigmata e.g., Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp., Varroa spp.

From the order Actineda (Prostigata) and Acarida (Astigmata) e.g., Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

The active ingredients of the formula (I) according to the invention are also suitable for controlling arthropods that infest agricultural livestock, such as cattle, sheep, goats, horses, hogs, donkeys, camels, buffalo, rabbits, chickens, turkeys, ducks, geese and bees; other pets, such as dogs, cats, caged birds, aquarium fish; and so-called test animals, such as hamsters, guinea pigs, rats and mice. By controlling these arthropods, deaths and reduction in productivity (for meat, milk, wool, hides, eggs, honey, etc.) should be reduced, making possible more economic and easier animal husbandry through the use of the active compounds according to the invention.

The active ingredients according to the invention are used in the veterinary sector in the customary manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear tags, tail tags, limb bands, halters, marking devices and the like.

When used for cattle, poultry, pets and the like, the active ingredients of the formula (1) can be used as formulations (for example powders, emulsions, free-flowing agents) that contain the active ingredients in an amount of 1 to 80% w/w, directly or after 100- to 10,000-fold dilution, or they can be used as a chemical bath.

It has furthermore been found that the compounds according to the invention exhibit a strong insecticidal effect against insects that destroy industrial materials.

The following insects are listed as examples and as being preferred—but without being limited to such:

Beetles such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec.

Tryptodendron spec. Apate monachus, Bostrychus capucins, Heterobostrychus brunneus, Sinoxylon spec. Dinoderus minutus;

Hymenoptera such as Sirex juvencus, Urocerus gigas, Urocerus gigas taignus, Urocerus augur;

Termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus;

Silverfish such as Lepisma saccharina.

Industrial materials in the present context are to be understood as meaning non-living materials, such as, preferably, plastics, adhesives, glues, papers and cardboard, leather, wood and processed wood products and coating materials.

Particularly preferably, wood and processed wood products are materials to be protected from insect infestation.

Wood and processed wood products that can be protected by the material according to the invention or mixtures comprising such are to be understood to be, for example: building timber, wooden beams, railway ties, bridge components, boat jetties, wooden vehicles, boxes, pallets, containers, telegraph poles, wood paneling, wooden windows and doors, plywood, chipboard, joinery or wooden products which are used quite generally in house-building or in building joinery.

The active ingredients can be used as such, in the form of concentrates or in generally customary formulations, such as powders, granulates, solutions, suspensions, emulsions or pastes.

The formulations mentioned can be prepared in a known manner per se, for example by mixing the active ingredients with at least one solvent or diluent, emulsifier, dispersing agent and/or binder or fixing agent, a water repellent, if appropriate, siccatives and UV stabilizers and if appropriate dyes and pigments, and also other auxiliary processing agents.

The insecticidal agents or concentrates used for the preservation of wood and wood-derived timber products comprise the active ingredient according to the invention in a concentration of 0.0001 to 95% w/w, in particular 0.001 to 60% w/w.

The amount of the materials or concentrates used depends on the nature and occurrence of the insects and on the medium. The optimum amount employed can be determined by a series of tests for each application. In general, however, it is sufficient to use 0.0001 to 20% w/w, preferably 0.001 to 10% w/w, of the active ingredient, depending on the material to be preserved.

Solvents and/or diluents can be organic chemical solvents or solvent mixtures and/or oily or oil-like organic chemical solvents or solvent mixtures of low volatility and/or polar organic chemical solvents or solvent mixtures and/or water, and, if appropriate, an emulsifier and/or wetting agent.

Organic chemical solvents which are preferably used are oily or oil-like solvents having a relative evaporation rate above 35 and a flashpoint above 30° C., preferably above 45° C. Such oily or oil-like water-insoluble solvents of low volatility can be appropriate mineral oils or aromatic fractions thereof, or solvent mixtures containing mineral oils, preferably solvent naphtha, petroleum and/or alkyl benzene.

Mineral oils having a boiling range of 170° C. to 220° C., solvent naphtha having a boiling range of 170° C. to 220° C., spindle oil having a boiling range of 250° C. to 350° C., petroleum or aromatics having a boiling range of 160° C. to 280° C., turpentine oil and the like, are advantageously used.

In a preferred embodiment, liquid aliphatic hydrocarbons having a boiling range of 180° C. to 210° C. or high-boiling mixtures of aromatic and aliphatic hydrocarbons having a boiling range of 180° C. to 220° C. and/or spindle oil and/or monochloronaphthalene, preferably α-monochloronaphthalene, are used.

The organic oily or oil-like solvents of low volatility that have a relative evaporation rate over 35 and a flashpoint over 30° C., preferably over 45° C., can be replaced in part by organic chemical solvents of high or medium volatility, provided that the solvent mixture likewise has a relative evaporation rate over 35 and a flashpoint above 30° C., preferably over 45° C., and that the insecticide/fungicide mixture is soluble or emulsifiable in this solvent mixture.

According to a preferred embodiment, part of the organic chemical solvent or solvent mixture is replaced by an aliphatic polar organic chemical solvent or solvent mixture. Aliphatic organic chemical solvents containing hydroxyl and/or ester and/or ether groups, such as glycol ethers, esters and the like, are preferably used.

Organic chemical binders that are used in the context of the present invention are synthetic resins and/or binding drying oils that are known per se, water-dilutable and/or are soluble or dispersible or emulsifiable in the organic chemical solvents employed, in particular binders consisting of or comprising an acrylate resin, a vinyl resin, for example polyvinyl acetate, polyester resin, polycondensation or polyaddition resin, polyurethane resin, alkyd resin or modified alkyd resin, phenolic resin, hydrocarbon resin, such as indene-cumarone resin, silicone resin, drying vegetable oils and/or drying oils and/or physically drying binders based on a natural and/or synthetic resin.

The synthetic resin used as the binder can be employed in the form of an emulsion, dispersion or solution. Bitumen or bituminous substances can also be used as binders in an amount of up to 10% w/w. Dyestuffs, pigments, water-repelling agents, odor correctants and inhibitors or anticorrosive agents and the like which are known per se can additionally be employed.

It is preferred according to the invention for the composition or concentrate to comprise, as the organic chemical binder, at least one alkyd resin or modified alkyd resin and/or one drying vegetable oil. Alkyd resins having an oil content of more than 45% w/w, preferably 50 to 68% w/w, are preferably used according to the invention.

All or some of the binder mentioned can be replaced by a fixing agent (mixture) or a softener (mixture). These additives are intended to prevent evaporation of the active compounds and crystallization or precipitation. They preferably replace 0.01 to 30% of the binder (based on 100% of the binder used).

The softeners originate from the chemical classes of phthalic acid esters, such as dibutyl, dioctyl or benzyl butyl phthalate, phosphoric acid esters, such as tributyl phosphate, adipic acid esters, such as di-(2-ethylhexyl) adipate, stearates, such as butyl stearate or amyl stearate, oleates, such as butyl oleate, glycerol ethers or higher molecular weight glycol ethers, glycerol esters and p-toluene sulfonic acid esters.

Fixing agents are based chemically on polyvinyl alkyl ethers, such as polyvinyl methyl ether or ketones, such as benzophenone or ethylene benzophenone.

Water, in particular, can be used as a solvent or diluent, optionally, mixed with one or more of the aforementioned organic chemical solvents or diluents, emulsifiers or dispersing agents.

Particularly effective wood preservation is achieved by impregnation processes done on a large industrial scale, for example, vacuum, double-vacuum or pressure processes.

The ready-to-use agents can also comprise other insecticides, if appropriate, and also one or more fungicides, if appropriate.

The insecticides and fungicides listed in WO 94/29268 are additionally considered preferred mixing partners. The compounds named in this document are an explicit part of the present filing.

Especially preferred mixing partners include insecticides, such as chloropyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyfenozide, triflumuron, clothianidin, spinosad, tefluthrin,

and fungicides such as epoxyconazole, hexaconazole, azaconazole, propiconazole, tebuconazole, cyproconazole, metconazole, imazalil, dichlor(fluanide, tolylfluanide, 3-iodine-2-propinyl-butylcarbamate, N-octyl-isothiazolin-3-one and 4,5-dichloro-N-octylisothiazolin-3-one.

The compounds according to the invention can at the same time be employed for protecting objects which come into contact with salt water or brackish water, such as hulls, screens, nets, structures, moorings and signalling systems, against fouling.

Fouling by sessile Oligochaeta, such as fan worms, and by mollusks and species from the Lepadomorpha suborer (goose barnacles), such as various Lepas and Scalpellum species, or by species from the Balanomorpha suborder (acorn barnacles), such as Balanus or Pollicipes species, increases the frictional drag of ships and, as a consequence, results in a marked increase in operational costs owing to higher energy consumption and additionally frequent time in dry dock.

Apart from fouling by algae, for example Ectocarpus sp. and Ceramium sp., fouling by sessile Entomostraca groups, which are grouped under Cirripedia (cirriped crustaceans), is of particular importance.

Surprisingly, it has now been found that the compounds according to the invention, alone or in combination with other active compounds, have an outstanding antifouling effect.

Using the compounds according to the invention, alone or in combinatin with other active ingredients, allows the use of heavy metals such as in bis-(trialkyltin) sulphides, tri-n-butyltin laurate, tri-n-butyltin chloride, copper (I) oxide, triethyltin chloride, tri-n-butyl(2-phenyl-4-chlorophenoxy)tin, tributyltin oxide, molybdenum disulphide, antimony oxide, polymeric butyl titanate, phenyl-(bispyridine)-bismuth chloride, tri-n-butyltin fluoride, manganese ethylene bisthiocarbamate, zinc dimethyl dithiocarbamate, zinc ethylene bisthiocarbamate, zinc salts and copper salts of 2-pyridinethiol 1-oxide, bisdimethyldithiocarbamoylzinc ethylene-bisthiocarbamate, zinc oxide, copper (I) ethylene-bisdithiocarbamate, copper thiocyanate, copper naphthenate and tributyltin halides to be dispensed with, or the concentration of these compounds substantially reduced.

If appropriate, the ready-to-use antifouling paints can additionally comprise other active ingredients, preferably algicides, fungicides, herbicides, molluscicides, or other antifouling active ingredients.

Preferably suitable combination partners with the antifouling agents according to the invention are:

Algicides such as 2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;

Fungicides such as benzo[b]thiophenecarboxylic acid cyclohexylamide S,S-dioxide, dichlofluanid, fluorfolpet, 3-iodo-2-propinyl butylcarbamate, tolylfluanid; and azoles such as azaconazole, cyproconazole, epoxyconazole, hexaconazole, metconazole, propiconazole and tebuconazole;

Molluscicides such as fentin acetate, metaldehyde, methiocarb, niclosamid, thiodicarb and trimethacarb; iron chelate,

or conventional anti-fouling active ingredients such as 4,5-dichloro-2-octyl-4-isothiazolin-3-one, diiodomethylparatryl sulphone, 2-(N,N-dimethylthiocarbamoyl-thio)-5-nitrothiazyl, potassium, copper, sodium and zinc salts of 2-pyridinethiol 1-oxide, pyridine-triphenylborane, tetrabutyldistannoxane, 2,3,5,6-tetrachloro-4-(-methylsulphonyl)-pyridine, 2,4,5,6-tetrachloroisophthalonitrile, tetramethylthiuram disulphide and 2,4,6-trichlorophenylmaleinmide.

The antifouling compositions used contain the active ingredient of the invention of the compounds of the invention [sic] in a concentration of 0.001 to 50% w/w, in particular 0.01 to 20% by weight.

Moreover, the antifouling compositions contain conventional components such as those described in Ungerer, Chem. Ind. 1985, 37, 730-732 and Williams, Antifouling Marine Coatings, Noyes, Park Ridge, 1973.

Besides algicidal, fungicidal, molluscicidal active ingredients and insecticidal active ingredients according to the invention, antifouling paints contain, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/ styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants, which are preferably insoluble in salt water. Paints may furthermore comprise materials such as colophonium to allow controlled release of the active compounds. Furthermore, the paints may comprise softeners, modifiers which affect the rheological properties and other conventional components. The compounds of the invention may also be incorporated into self-polishing antifouling systems.

The active ingredients are also suitable for controlling animal pests, in particular insects, arachnids and mites, which are found in enclosed spaces such as dwellings, factory floors, offices, vehicle passenger cabins and the like. They can be used alone in household insecticide products for controlling these pests or in combination with other active ingredients and adjuvants. They are effective for sensitive and resistant species as well as for all life stages. These pests include:

From the order Scorpionidae, e.g., Buthus occitanus.

From the order Acaria, e.g., Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order Araneae, e.g., Aviculariidae, Araneidae.

From the order Opiliones, e.g., Pseudoscorpiones chelifer, Pseudoscorpiones cheiridium, Opiliones phalangium.

From the order Isopoda, e.g., Oniscus asellus, Porcellio scaber.

From the order Diplopoda, e.g., Blaniulus guttulatus, Polydesmus spp.

From the order Chilopoda, e.g., Geophilus spp.

From the order Zygentoma, e.g., Ctenolepisma spp., Lepisma saccharina, Lepismodes inquilinus.

From the order Blattaria, e.g., Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order Saltatoria, e.g., Acheta domesticus.

From the order Dermaptera, e.g., Forficula auricularia.

From the order Isoptera, e.g., Kalotermes spp., Reticulitermes spp.

From the order Psocoptera, e.g., Lepinatus spp., Liposcelis spp.

From the order Coleptera, e.g. Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order Diptera, e.g., Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order Lepidoptera, e.g., Achroia grisella, Galleria mellonella, Plodia interpunctella, Tinea cloacella, Tinea pellionella, Tineola bisselliella.

From the order Siphonaptera, e.g., Ctenocephalides canis, Ctenocephalides felis, Pulex irritans, Tunga penetrans, Xenopsylla cheopis.

From the order Hymenoptera, e.g., Camponotus herculeanus, Lasius fuliginosus, Lasius niger, Lasius umbratus, Monomorium pharaonis, Paravespula spp., Tetramorium caespitum.

From the order Anoplura, e.g., Pediculus humanus capitis, Pediculus humanus corporis, Phthirus pubis.

From the order Heteroptera, e.g., Cimex hemipterus, Cimex lectularius, Rhodinus prolixus, Triatoma infestans.

They are used in the household insecticides sector alone or in combination with other suitable active ingredients such as phosphoric acid esters, carbamates, pyrethroids, neo-nicotinoids, growth regulators or active ingredients from other known classes of insecticides.

They are applied in aerosols, unpressurized spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granulates or dusts, in baits for spreading or in bait stations.

### PREPARATION EXAMPLES

### Example 1

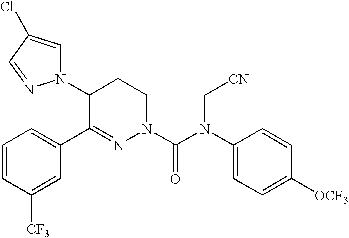 (Process (b))

Put 0.5 g triphosgene into 30 ml methylene chloride and cool to 0° C. and, while stirring, add a solution of 1.1 g (5 mMol) 4-trifluoromethoxy-phenyl-amino-acetonitrile in 25 ml methylene chloride and then add 0.7 ml triethylamine, drop by drop. Stir the mixture for 30 minutes at room temperature (approx. 20° C.). Then add a solution of 1.6 g (5 mMol) 4-(4-chloropyrazole-1-yl)-3-(trifluoromethylphenyl)-1,4,5,6-tetrahydropyridazine to 25 ml methylene chloride and then add to the mixture 0.7 ml triethylamine, drop by drop, while stirring, and stir the reaction mixture at room temperature for 15 hours. To process, wash with a saturated aqueous sodium hydrogen carbonate solution, separate the organic phase, dry with sodium sulfate and filter. Concentrate the filtrate under low pressure and separate the residue using column chromatography.

The resulting amorphous product is 0.68 g (24% of the theoretical) (5 mMol) N-cyanomethyl-4-(4-chloropyrazole-1-yl)-1-(4-trifluoromethoxy-phenylamino)-3-(trifluoromethyl-phenyl)-carbonyl-1,4,5,6-tetrahydropyridazine. logp (pH: 2.3): 4.22.

Analagous to Example. 1, and corresponding to the general description of the preparation processes of the invention, the compounds of the formula (I) listed in Table 1 below can, for example, also be prepared.

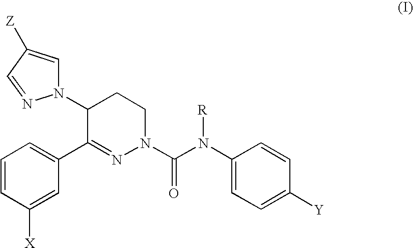

TABLE 1
Examples of compounds of the formula (I)

| Ex. No. | R | X | Y | Z | Phys. Data |
|---|---|---|---|---|---|
| 2 | CH₂CN | CF₃ | Cl | Cl | |
| 3 | CH₂CN | CF₃ | Br | Cl | |
| 4 | CH₂CN | CF₃ | Cl | CN | |
| 5 | CH₂CN | CF₃ | Br | CN | |
| 6 | CH₂CN | CF₃ | OCF₃ | CN | |
| 7 | COOCH₃ | Cl | OCF₃ | Cl | logP = 4.45^{a)} |
| 8 | COOCH₃ | Cl | OCF₃ | CN | logP = 3.89^{a)} |
| 9 | COOCH₃ | Cl | SCF₃ | CN | logP = 4.18^{a)} |
| 10 | CH₂CN | Cl | OCF₃ | CN | logP = 3.60^{a)} |
| 11 | CH₂CN | CN | OCF₃ | Cl | |
| 12 | CH₂CN | CN | OCF₃ | CN | |
| 13 | CH₂CN | Br | OCF₃ | Cl | |
| 14 | CH₂CN | Br | OCF₃ | CN | |
| 15 | CH₂CH₂CN | CF₃ | OCF₃ | Cl | |
| 16 | CH₂CH₂CN | CF₃ | OCF₃ | CN | |
| 17 | CH₂OCH₃ | CF₃ | OCF₃ | Cl | |
| 18 | CH₂OCH₃ | CF₃ | OCF₃ | CN | |
| 19 | CH₃ | CF₃ | OCF₃ | Cl | |
| 20 | CH₃ | CF₃ | OCF₃ | CN | |
| 21 | C₂H₅ | CF₃ | OCF₃ | Cl | |
| 22 | C₂H₅ | CF₃ | OCF₃ | CN | |
| 23 | CH₂CN | CF₃ | CF₃ | Cl | |
| 24 | CH₂CN | CF₃ | CF₃ | CN | |
| 25 | CH₂CN | F | OCF₃ | Cl | |
| 26 | CH₂CN | F | OCF₃ | CN | |
| 27 | CH₂CN | Cl | OCF₃ | Cl | |
| 28 | CH₂CN | Cl | OCF₃ | CN | |
| 29 | CH₂CN | Cl | CF₃ | Cl | |
| 30 | CH₂CN | Cl | CF₃ | CN | |
| 31 | CH₂CN | CN | CF₃ | Cl | |
| 32 | CH₂CN | CN | CF₃ | CN | |
| 33 | CH₂CN | Br | CF₃ | Cl | |
| 34 | CH₂CN | Br | CF₃ | CN | |
| 35 | CH₂CN | CF₃ | SCF₃ | Cl | |
| 36 | CH₂CN | CF₃ | SCF₃ | CN | |

Determine the logP values given in the table as specified in EEC directive 79/831 ex V.A8 using HPLC (high performance liquid chromatography) in a reversed e column (C 18). Temperature: 43° C.

(a) eluents for determination in the acid range: 0.1% aqueous phosphoric acid, acetonitrile, linear gradient of 10% acetonitrile to 90% acetonitrile—corresponding measurement results are marked with an a) in Table 1.

(b) eluents for determination in the neutral range: 0.01-molar aqueous phosphate buffer solution, acetonitrile, linear gradient of 10% acetonitrile to 90% acetonitrile—corresponding measurement results are marked with a b) [sic] in Table 1.

Calibration is done using unbranched alkane-2-ones (with 3 to 16 carbon atoms), whose logP values are known (determination of the logP values using the retention times via linear interpolation between two sequential ketones).

The lambda max values were determined using the UV spectrums of 200 nm to 400 nm in the maxima of the chromatographical signals.

Preparation of Starting Materials of The formula (II)

### Example (II-1)

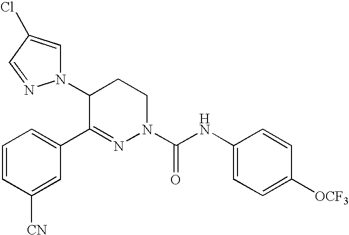

Drop-by-drop add a solution of 0.89 g (4.4 mMol) 4-trifluoromethoxy phenyl isocyanate in 10 ml acetonitrile to a solution of 1.27 g (4.4 mMol) 3-(3-cyanophenyl)-4-(4-chloropyrazol-1-yl)-1,4,5,6-tetrahydro-pyridazine in 30 ml acetonitrile at room temperature and stir the mixture for 6 hours at room temperature. Then filter the precipitated product and concentrate the filtrate at low pressure. Triturate the residue with ethanol, siphon after crystallization and rewash with ethanol.

This produces 0.44 g (20% of the theoretical) 3-(3-cyanophenyl)-4-(4-chloropyrazol-1-yl)-1-(4-trifluoromethoxy-phenylamino)-carbonyl-1,4,5,6-tetrahydropyridazine in the form of colorless crystals with the logp (pH2) =4.08 and a melting point of 216° C.

Preparation of Starting Materials of The formula (IV)

### Example (IV-1)

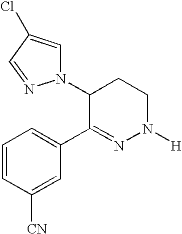

Add 1.7 g (35 mMol) hydrazine hydrate to a solution of 5.3 g (17.2 mMol) 3′-cyano-2-(4-chloro-pyrazole-1-yl)-4-chlorobutyrophenone in 50 ml ethanol and stir the mixture for 16 hours at room temperature. Next, distill the solvent in a vacuum, then add water to the residue and shake with ethyl acetate. Separate the organic phase, dry over sodium sulfate and concentrate in a vacuum.

This produces 3.8 g (78% of the theoretical) 3-(3-cyanophenyl)-4-(4-chloropyrazol-1-yl)-1,4,5,6-tetrahydropyridazine in the form of a colorless powder with the logp (pH2)=2.29 and a melting point of 171° C.

Preparation of Starting Materials of The formula (IX)

### Example (IX-1)

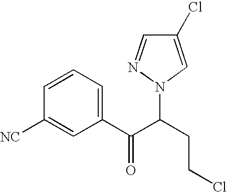

Drop by drop, at −5° C., add a solution of 14.3 g (0.05 Mol) 3′-cyano-2-bromo-4-chlorobutyrophenone in 20 ml acetonitrile to a mixture of 7 g (0.05 Mol) 4-chloropyrazole-hydrochloride, 13.8 g (0.1 Mol) potassium carbonate and 60 ml acetonitrile, and then stir the mixture for 18 hours at room temperature. Then add water to the reaction mixture and extract using ethyl acetate. Separate the organic phase, dry over sodium sulfate and concentrate in a vacuum. Triturate the residue with isopropanol, let sit at room temperature for 16 hours and then siphon the precipated product.

This produces 3.8 g (25% of the theoretical) 3′-cyano-2-(4-chloropyrazole-1-yl)-4-chlorobutyrophenone in the form of a beige powder with the logp (pH2)=2.95.

Preparation of Starting Materials of The formula (X)

### Example (X-1)

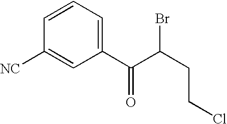

Add 0.1 g aluminium chloride to a solution of 32.7 g (0.15 Mol) 3′-cyano-4-chloro-butyrophenone in 150 ml methylene chloride [sic] and then add 25.6 g (0.16 Mol) bromine, drop by drop. Stir the mixture for 16 hours at room temperature and then direct nitrogen through the reaction mixture to remove the hydrogen bromide. While stirring, add water and then add saturated sodium bicarbonate solution until the aquaeous phase is neutral. Separate the organic phase, dry over sodium sulfate, filter and concentrate in a vacuum. Triturate the residue with petroleum ethers and siphon the product after crystallization.

This produces 40.8 g (95% of the theoretical) 3′-cyano-2-bromo-4-chlorobutyrophenone in the form of a beige powder with the logp (pH2)=−3.09.

### USE EXAMPLES

### Example A

Heliothis armigera Test

| | |
|---|---|
| Solvent: | 7 parts per weight dimethyl formamide |
| Emulsifier: | 2 parts per weight alkylaryl polyglycol ether |

To prepare one of the appropriate active ingredient preparations, mix 1 part by weight of the active ingredient with the indicated amount of solvent and emulsifier and dilute the concentrate with emulsifier-containing water to the desired concentration.

Treat soybean sprouts (Glycine max) by dipping into the active ingredient preparation of the desired concentration and infesting with Heliothis armigera larvae while the leaves are still damp.

After the desired time, determine the death rate in %. For these purposes 100% means that all the larvae on the leaves were killed; 0% means that no larvae were killed.

In this test the compound of preparation example 1, for example, exhibited good efficacy.

TABLE A
Plant-damaging Insects
Heliothis armigera Test

| | Concen- | |
|---|---|---|
| | tration | Death |
| | of Active | rate |
| | Ingredient | in % |
| Active Ingredients | in ppm | after 7d |
| 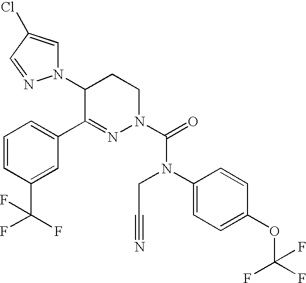 | 500 | 100 |

### Example B

Phaedon Larvae Test

| | |
|---|---|
| Solvent: | 7 parts per weight dimethyl formamide |
| Emulsifier: | 2 parts per weight alkylaryl polyglycol ether |

To prepare one of the appropriate active ingredient preparations, mix 1 part by weight of the active ingredient with the indicated amount of solvent and emulsifier and dilute the concentrate with emulsifier-containing water to the desired concentration.

Treat cabbage leaves (Brassica oleracea) by dipping into the active ingredient preparation of the desired concentration and infesting with larvae of the mustard beetle (Phaedon cochleariae) while the leaves are still damp.

After the desired time, determine the death rate in %. For this purpose 100% means that all the beetle larvae on the leaves were killed; 0% means that no beetle larvae were killed.

In this test the compound of preparation example 1, for example, exhibited good efficacy.

TABLE B
Plant-damaging Insects
Phaedon Larvae Test

| | Concen- | |
|---|---|---|
| | tration | Death |
| | of Active | rate |
| | Ingredient | in % |
| Active Ingredients | in ppm | after 7d |
| 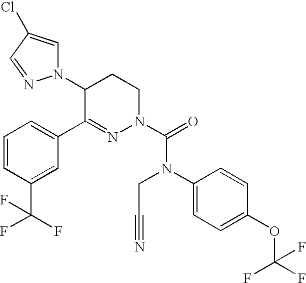 | 500 | 100 |

### Example C

Plutella Test

| | |
|---|---|
| Solvent: | 7 parts per weight dimethyl formamide |
| Emulsifier: | 2 parts per weight alkylaryl polyglycol ether |

To prepare one of the appropriate active ingredient preparations, mix 1 part by weight of the active ingredient with the indicated amount of solvent and emulsifier and dilute the concentrate with emulsifier-containing water to the desired concentration.

Treat cabbage leaves (Brassica oleracea) by dipping into the active ingredient preparation of the desired concentration and infesting with larvae of the diamondback moth (Plutella xylostella) while the leaves are still damp.

After the desired time, determine the death rate in %. For these purposes 100% means that all the larvae on the leaves were killed; 0% means that no larvae were killed.

In this test the compound of preparation example 1, for example, exhibited good efficacy.

TABLE C
Plant-damaging Insects
Plutella Test

| | Concen- | |
|---|---|---|
| | tration | Death |
| | of Active | rate |
| | Ingredient | in % |
| Active Ingredients | in ppm | after 7d |
| 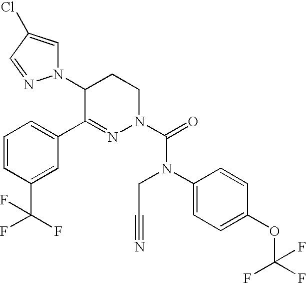 | 500 | 100 |

### Example D

Spodotera exigua Test

| | |
|---|---|
| Solvent: | 7 parts per weight dimethyl formamide |
| Emulsifier: | 2 parts per weight alkylaryl polyglycol ether |

To prepare one of the appropriate active ingredient preparations, mix 1 part by weight of the active ingredient with the indicated amount of solvent and emulsifier and dilute the concentrate with emulsifier-containing water to the desired concentration.

Treat cabbage leaves (Brassica oleracea) by dipping into the active ingredient preparation of the desired concentration and infesting them with larvae of the beet armyworm (Spodoptera exigua) while the leaves are still damp.

After the desired time, determine the death rate in %. For these purposes 100% means that all the larvae on the leaves were killed; 0% means that no larvae were killed.

In this test the compound of preparation example 1 exhibited good efficacy.

TABLE D
Plant-damaging Insects
Spodotera exigua Test

| | Concen- | |
|---|---|---|
| | tration | Death |
| | of Active | rate |
| | Ingredient | in % |
| Active Ingredients | in ppm | after 7d |
| 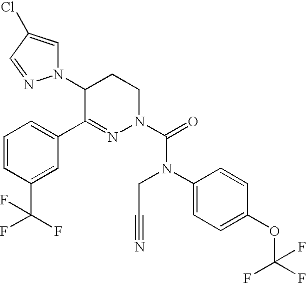 | 500 | 100 |

### Example E

Spodoptera frugiperda Test

| | |
|---|---|
| Solvent: | 7 parts per weight dimethyl formamide |
| Emulsifier: | 2 parts per weight alkylaryl polyglycol ether |

To prepare one of the appropriate active ingredient preparations, mix 1 part by weight of the active ingredient with the indicated amount of solvent and emulsifier and dilute the concentrate with emulsifier-containing water to the desired concentration.

Treat cabbage leaves (Brassica oleracea) by dipping into the active ingredient preparation of the desired concentration and infesting them with larvae of the fall armyworm (Spodoptera frugiperda) while the leaves are still damp.

After the desired time, determine the death rate in %. For these purposes 100% means that all the larvae on the leaves were killed; 0% means that no larvae were killed.

In this test the compound of preparation example 1, for example, exhibited good efficacy.

TABLE E
Plant-damaging Insects
Spodoptera frugiperda Test

| | Concen- | |
|---|---|---|
| | tration | Death |
| | of Active | rate |
| | Ingredient | in % |
| Active Ingredients | in ppm | after 7d |
| 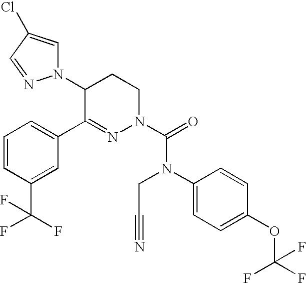 | 500 | 100 |

### Example F

Diabrotica balteata Test (Larvae in the Soil)

Concentration Limits Test/Soil Insects—Treatment of Transgenic Plants

| | |
|---|---|
| Solvent: | 3 parts per weight dimethyl formamide |
| Emulsifier: | 1 part per weight alkylaryl polyglycol ether |

To prepare one of the appropriate active ingredient preparations, mix 1 part by weight of the active ingredient with the indicated amount of solvent, add the indicated amount of emulsifier, and dilute the concentrate with water to the desired concentration.

Mix the active ingredient preparation deeply into the soil. For these purposes, the concentration of the active ingredient in the preparation matters very little, the only decisive factor is the amount of active ingredient per volume unit of soil, which is given in ppm (mg/L). Put the soil into 0.25 L pots and let same stand at 20° C.

Immediately after preparing the soil, put 5 pre-sprouted corn kernals of the YIELD GARD (Monsanto Co., USA, registered trademark) variety into each pot. Two days later put the respective test insects into the treated soil. After another 7 days determine the degree of efficacy of the active ingredient by counting the visible corn plants (1 plant=20% efficacy).

### Example G

Heliothis virescens Test Treatment of Transgenic Plants

| | |
|---|---|
| Solvent: | 7 parts by weight |
| Emulsifier: | 1 part per weight alkylaryl polyglycol ether |

To prepare one of the appropriate active ingredient preparations, mix 1 part by weight of the active ingredient with the indicated amount of solvent and the indicated amount of emulsifier, and dilute the concentrate with water to the desired concentration.

Treat soybean sprouts (i Glycine max) of the Roundup Ready (registered trademark of the Monsanto Co., USA) variety by dipping them into the active ingredient preparation of the desired concentration and infesting them with the tobacco bud worm Heliothis virescens while the leaves are still damp.

After the desired time, determine the death rate of the insects. For these purposes 100% means that all the larvae on the leaves were killed; 0% means that no larvae were killed.

What is claimed is:

1-11. (canceled)

12. A tetrahydropyridazine derivative of formula (I) 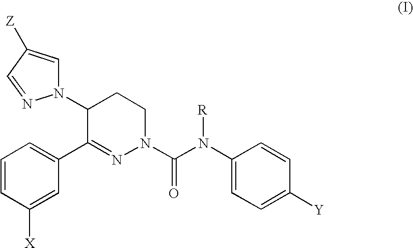
wherein
R represents optionally substituted alkyl, alkoxycarbonyl, alkenyl, alkynyl, cycloalkyl, or cycloalkylaklyl,
X represents cyano, halogen, halogenoalkyl, halogenoalkoxy, aklylthio, alkylsulfinyl, alkylsulfonyl, halogenoalkylthio, halogenoalkylsulfinyl, or halogenoalkylsulfonyl,
Y represents cyano, halogen, halogenoalkyl, halogenoalkoxy, halogenoalkylthio, halogenoalkylsulfinyl, or halogenoalkylsulfonyl, and
Z represents hydrogen, hydroxy, nitro, cyano, carbamoyl, halogen, alkyl, alkoxy, alkylthio, halogenoalkyl, halogenoalkoxy, halogenoalkylthio, alkoxycarbonyl, alkylaminocarbonyl, or dialkylaminocarbonyl.

13. A tetrahydropyridazine derivative of formula (I) according to claim 12 wherein
R represents optionally cyano-, carboxy-, carbamoyl-, halogen-, C₁-C₄ alkoxy-, C₁-C₄ alkylcarbonyl-, or C₁-C₄ alkoxycarbonyl-substituted alkyl having 1 to 6 carbon atoms; alkoxycarbonyl having up to 6 carbon atoms; optionally cyano-, or halogen-substituted alkenyl or alkynyl, each having 2 to 6 carbon atoms; or optionally cyano-, halogen-, or C₁-C₄ alkyl-substituted cycloalkyl or cycloalkylalkyl, each having 3 to 6 carbon atoms in the cycloalkyl group and, optionally, 1 to 4 carbon atoms in the alkyl moiety,
X represents cyano, halogen, C₁-C₄ halogenoalkyl, C₁-C₄ halogenoalkoxy, C₁-C₄ alkylthio, C₁-C₄ alkylsulfinyl, C₁-C₄ alkylsulfonyl; C₁-C₄ halogenoalkylthio, C₁-C₄ halogenoalkylsulfinyl, or C₁-C₄ halogenoalkylsulfonyl, whereby each halogeno-alkyl group contains 1 to 5 same or different halogen substituents selected from the group consisting of fluorine, chlorine, and bromine,
Y represents cyano, halogen, C₁-C₄ halogenoalkyl, C₁-C₄ halogenoalkoxy, C₁-C₄ halogenoalkylthio, C₁-C₄ halogenoalkylsulfinyl, or C₁-C₄ halogenoalkylsulfonyl, wherein each halogenoalkyl group contains 1 to 5 same or different halogen substituents selected from the group consisting of fluorine, chlorine, and bromine, and
Z represents hydrogen, hydroxy, nitro, cyano, carbamoyl, halogen, C₁-C₄ alkyl, C₁-C₄ alkoxy, C₁-C₄ alkylthio; C₁-C₄ halogenoalkyl, C₁-C₄ halogenoalkoxy, or C₁-C₄ halogenoalkylthio, wherein each halogenoalkyl group contains 1 to 5 same or different halogen substituents selected from the group consisting of fluorine, chlorine, and bromine; or C₁-C₄ alkoxycarbonyl, C₁-C₄ alkyl-amino-carbonyl, or C₁-C₄ dialkyl-aminocarbonyl.

14. A tetrahydropyridazine derivative of formula (I) according to claim 12 wherein
R represents optionally cyano-, carboxy-, carbamoyl-, fluorine-, chlorine-, methoxy-, ethoxy-, n- or i-propoxy-, acetyl-, propionyl-, n- or i-butyroyl-, methoxycarbonyl-, ethoxycarbonyl-, or n- or i-propoxycarbonyl substituted methyl, ethyl, n- or i-propyl, or n-, i-, or s-butyl; optionally cyano-, fluorine-, and/or chlorine- substituted ethenyl, propenyl, butenyl, ethynyl, propynyl, or butynyl; or optionally cyano-, fluorine-, chlorine-, methyl-, or ethyl-substituted cyclopropyl, cyclobutyl, cyclopentyl, cyclohexyl, cyclopropylmethyl, cyclobutylmethyl, cyclopentylmethyl, or cyclohexylmethyl,
X represents cyano, fluorine, chlorine, bromine, difluoromethyl, trifluoromethyl, dichloromethyl, trichloromethyl, chlorodifluoromethyl, fluorodichloromethyl, difluoromethoxy, trifluoromethoxy, chlorodifluoromethoxy, methylthio, methylsulfinyl, methylsulfonyl, trifluoromethylthio, trifluoromethylsulfinyl, or trifluoromethylsulfonyl,
Y represents cyano, fluorine, chlorine, bromine, difluoromethyl, trifluoromethyl, dichloromethyl, trichloromethyl, chlorodifluoromethyl, fluorodichloromethyl, difluoromethoxy, trifluoromethoxy, chlorodifluoromethoxy, trifluoromethylthio, trifluoromethylsulfinyl, or trifluoromethylsulfonyl, and
Z represents hydrogen, cyano, carbamoyl, chlorine, bromine, methyl, methoxy, methylthio, trifluoromethyl, trifluoromethoxy, trifluoromethylthio, methoxycarbonyl, ethoxycarbonyl, methylaminocarbonyl, ethylaminocarbonyl, or dimethylaminocarbonyl.

15. A tetrahydropyridazine derivative of formula (I) according to claim 12 wherein
R represents optionally cyano-, fluorine-, chlorine-, methoxy-, ethoxy-, acetyl-, propionyl-, methoxycarbonyl-, ethoxycarbonyl-, or n- or i-propoxycarbonyl-substituted methyl, ethyl, or n- or i-propyl; optionally cyano-, fluorine-, and/or chlorine-substituted propenyl, butenyl, ethynyl, propynyl, or butynyl; or optionally fluorine-, chlorine-, or methyl-substituted cyclopropyl, cyclobutyl, cyclopentyl, cyclohexyl, cyclopropylmethyl, cyclobutylmethyl, cyclopentylmethyl, or cyclohexylmethyl,
X represents cyano, fluorine, chlorine, bromine, trifluoromethyl, difluoromethoxy, trifluoromethoxy, chlorodifluormethoxy, methylthio, methylsulfinyl, methylsulfonyl, trifluoromethylthio, trifluoromethylsulfinyl, or trifluoromethylsulfonyl,
Y represents cyano, fluorine, chlorine, bromine, trifluoromethyl, difluoromethoxy, trifluoromethoxy, chlorodifluoromethoxy, trifluoromethylthio, trifluoromethylsulfinyl, or trifluoromethylsulfonyl, and
Z represents hydrogen, cyano, carbamoyl, chlorine, bromine, methyl, methoxy, methylthio, trifluoromethyl, trifluoromethoxy or trifluoromethylthio.

16. A tetrahydropyridazine derivative of formula (I) according to claim 12 wherein R represents cyanomethyl and X represents trifluoromethyl.

17. A process for preparing tetrahydropyridazine derivatives of formula (I) according to claim 12 comprising converting
(a) a tetrahydropyridazine of formula (II) 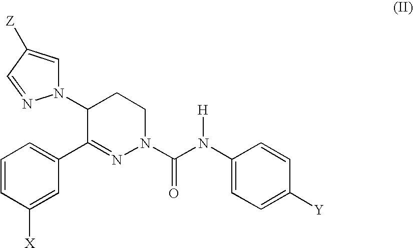
wherein X, Y, and Z have the meanings given for formula (I) in claim 12,
with a compound of formula (III)
X¹—R (III)
wherein
X¹ represents halogen or the grouping R—O—SO₂—O—, and
R has the meanings given for formula (I) in claim 11, optionally in the presence of one or multiple reaction adjuvants and optionally in the presence of one or multiple diluents,
or
(b) a tetrahydropyridazine of formula (IV) 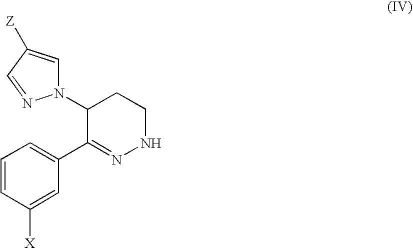
wherein X and Z have the meanings given for formula (I) in claim 12, with an N-substituted arylamine of formula (V) 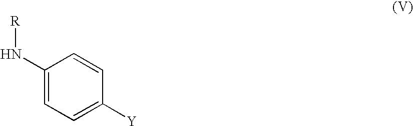
wherein R and Y have the meanings given for formula (I) in claim 12, in the presence of a reactive carbon dioxide derivative of formula (VI)
X²—CO—X² (VI)
wherein X² and X³ are the same or different and represent halogen,
alkoxy, halogenoalkoxy, phenoxy, or benzyloxy, optionally in the presence of one or multiple reaction adjuvants and optionally in the presence of one or multiple diluents,
or
(c) a tetrahydropyridazine of formula (IV) 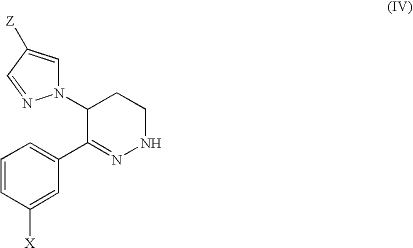
wherein X and Z have the meanings given for formula (I) in claim 12, with a carbamic acid derivative of formula (VII) 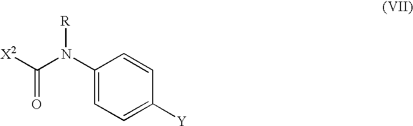
wherein
R and Y have the meanings given for formula (I) in claim 12, and
X² represents halogen, optionally in the presence of one or multiple reaction adjuvants and optionally in the presence of one or multiple diluents.

18. A pesticide comprising one or more compounds of formula (I) according to claim 12 and one or more extenders and/or surfactants.

19. A process for controlling animal pests comprising allowing one or more compounds of formula (I) according to claim 12 to act on pests and/or their habitat.

20. A process for preparing pesticides comprising mixing one or more compounds of formula (I) according to claim 12 with one or more extenders and/or surfactants.
